(19) United States
(12) Patent Application Publication
(10) Pub. No.: US 2018/0325720 A1
(43) Pub. Date: Nov. 15, 2018

(54) DISPOSABLE BAG TO COLLECT URINE AND OTHER BODY LIQUIDS

(71) Applicant: Fernanda Coelho BRITES PINTO, Rio de Janeiro (BR)

(72) Inventor: Fernanda Coelho BRITES PINTO, Rio de Janeiro (BR)

(21) Appl. No.: 16/032,395

(22) Filed: Jul. 11, 2018

Related U.S. Application Data
(63) Continuation-in-part of application No. 15/432,974, filed on Feb. 15, 2017.

(30) Foreign Application Priority Data
Jan. 4, 2017 (BR) BR1020170001733

(51) Int. Cl. A61F 5/451; A61F 5/44; B65D 75/00; B65D 33/00; B65D 33/25

(52) U.S. Cl. CPC A61F 5/451; A61F 5/4404; B65D 33/25; B65D 33/004; B65D 75/008

(57) ABSTRACT

A disposable bag to collect urine and other body liquids that is made from a simple construction and at low cost, allowing a comfortable and secure use, ensuring, after use, the appropriate hermetic sealing, allowing the gelled content inside the bag to assume a regular cylindrical-stem format, facilitating, with the assistance of the metric scale located outside the bag, to establish accurately the correspondence of the volume of liquid lost by the patient, in order that the precise body fluid balance of the patient can be measured.

### CROSS REFERENCE TO RELATED APPLICATION

This application is a continuation-in-part of U.S. application Ser. No. 15/432,974 filed Feb. 15, 2017, entitled, DISPOSABLE BAG FOR THE COLLECTION OF URINE, VOMIT, AND OTHERS, pending, the content of which is incorporated herein by reference, which claimed priority over Brazilian Patent Application No. BR102017 0001733 filed Jan. 4, 2017.

### FIELD OF THE INVENTION

Specifications of a patent of a disposable bag to collect urine and other body liquids, having gel inside, with a capacity to absorb liquid appropriate to the employment of the said bag, and which is preferentially designed for hospital use, and which can, after receiving urine or other body liquid inside, be kept stable in a vertical position, due to its structural characteristics, allowing the gel, by the absorption of the urine or other body liquid, to assume a regular cylindrical-stem form, which, with the assistance of a scale located on the outside of the said bag, allows the checking of the loss of liquid of the patient, and the undertaking of the appropriate body fluid balance of the patient, precisely controlling the volume of liquid to be administered, according to the loss of liquid.

### PRIOR ART

The document of patent U.S. Pat. No. 5,007,116 discloses a portable urinal that comprises of a storage bag for urine, made from an impermeable material, and a pouch, made from a permeable material, filled with a liquid absorbing agent, which, in contact with the urine, absorbs the urine, forming a gel. The said pouch breaks into fragments when the absorbing agent absorbs the urine. The portable urinal also comprises of a storage bag for urine, made from an impermeable material, with a mouth that covers the genitourinary area of a man or woman when in use. The portable urinal also comprises of a storage bag for urine, made from an impermeable material, provided with a fastener with a grooved channel, located inside the front part of the storage bag for urine, at a suitable position thereof, and by a ridge, located on the inside the rear part of the storage bag for urine, at a suitable position thereof, which is detachably inserted into the grooved channel in a snapping manner; the grooved channel and the ridge extending along the width of the storage bag for urine.

The document of patent U.S. Pat. No. 5,091,998 discloses a funnel device with a foldable directional tube, to facilitate directional urination by women, for health reasons and for convenience. The funnel has a generally oval rim and the body terminates at the narrow end, forming a drainage tube, which extends outwards.

The funnel rim is rounded, narrowing towards the rear and is generally shaped to engage the pubic area adjacent the urethra, ensuring that the urine expelled from the urethra will be collected in the funnel and directed away from the body of the user. The drainage tube is directed toward the widest portion (the front) of the funnel, at an angle for directing the urine flow away from the user's body when standing upright. Although the rim of the funnel is semi-rigid, the body of the funnel is preferably flexible, whereby the unit is collapsible to facilitate storage. The drainage tube of the funnel is made flexible by means of one or more circumferentially corrugated areas along its length. The flexing of the drainage tube at the corrugations also permits collapsing for compact storage, and furthermore assists in directing the flow of urine to a specific place of disposal.

The entire unit folds and is easily packaged for carrying in a purse or pocket. The unit may be completely disposable, when non-reusable flexible plastic materials are used, or may be re-usable, when latex or other washable fabric is used. A packet is preferably provided for carrying the device and the packet may be disposable or reusable, providing the materials of construction are washable and antiseptic. In all instances, the preferred drainage tube is a foldable plastic tube bent back over the funnel for compact storage, and bent away from the funnel to a desired orientation for directing the flow of urine during use.

The document of patent US2009/0056003 also discloses a personal urinal funnel comprising of a receptacle with angled walls, to minimize splashes, an opening to receive the urine and an orifice to dispense the urine through a discharge tube. The said opening may be oval.

The document of patent U.S. Pat. No. 5,356,398 discloses a plastic bag for the collection of body fluids having a funnel mounted at the upper internal part of the structure, with associated resiliently flexible, funnel structure, enabling the funnel to be manually opened and closed. The upper extremity of the funnel is sealed to the inner wall of the bag in a position which causes the upper extremity of the funnel to reside below a pair of closure lips formed at the top of the bag. The pair of lips located on the upper edges of the bag comprise of a pair of complementary sealing strips which permit secure closure. The outside of the bag is imprinted with a scale indicative of the volume of fluid collected, having an opaque white strip onto which needed information may be written. The sheet plastic of which the bag is formed may be either clear for general medical application to permit ease of inspection or opaque in the case of aircraft use. The bag also includes a valve that is sealed to a lower portion of the funnel to prevent the bag's contents from minor spills before the sealing strips securely seal the bag closed.

The document of patent U.S. Pat. No. 6,199,220 discloses a plastic urine bag comprising of a bag portion and a collar comprised of a single piece of plastic and having a top edge, a medial edge, two distal edges, and an inside face, in which a tab extends outward from each of the distal edges, having a lip that extends at least partially around the top edge, and also having two sets of ridges on the inside face of the collar. The ridges are positioned to form a closure mechanism when the collar is folded along the medial edge. In a preferred embodiment, the plastic collar adheres to a portion inside the part of the bag, and extends approximately one-fifth of the length of the bag portion. In yet another preferred embodiment, the plastic collar adheres to a portion inside the part of the bag, and extends between approximately one-eighth and approximately one-third of the length of the bag portion. In a preferred embodiment, the bag contains a composition comprised of a superabsorbent polymer, an odor control agent, and/or a decay catalyst. The superabsorbent polymer is preferably sodium polyacrylate. The odor control agent is preferably zeolite. The decay catalyst is preferably an enzyme found in yeast. In a preferred embodiment, the enzyme is selected from the group consisting of sucrase, zymase, maltase, lactase, hexosephosphatase, reductase, carboxylase, melibiase, and endotryptase. In a preferred embodiment, the percentage by weight of the superabsorbent polymer is situated between 0.1% to 99%; the percentage by weight of the odor control agent is situated between 0.1% to 25%, and the percentage by weight of the decay catalyst is between 0.1% to 15%. In yet another preferred embodiment, the percentage by weight of the superabsorbent polymer is 84%; the percentage by weight of the odor control agent is 15%, and the percentage by weight of the decay catalyst is 1%. Preferably, the composition comprises of approximately 0.0302 pounds of sodium polyacrylate, approximately 0.0055 pounds of zeolite, and 0.0003 pounds of yeast. In a preferred embodiment, the part of the bag and the plastic collar are comprised of a biodegradable material.

The document of patent U.S. Pat. No. 6,199,220 discloses a portable container for receiving human waste products, in particular, urine and fecal matter. In a first embodiment for micturition by males, the device is a cylinder having an outside layer made from a pliable, liquid impermeable plastic and an inside layer made from a soft, absorbent material. The device includes a pair of foldable flaps at the open mouth of the cylinder and a means to close the mouth of the cylinder for disposal after use. In a second embodiment for micturition by females, the device is generally rectangular with rounded corners having an outside layer made from a flexible, impermeable plastic and an inside layer made from a soft, absorbent material. A flap is attached to one end of the outside layer and a flexible shroud is attached to an opposing end of the outside layer, so that, after use, the flap is used to fold the device in half and the shroud is pulled over the layers for disposal. In a third embodiment for receiving solid waste, the device includes an inflatable, cylindrical chamber, having a liquid impermeable plastic bag placed over the chamber, the bottom of the bag having a layer of absorbent material chemically treated to reduce odor being disposed in the bottom of the bag, the mouth of the bag being retained to the outer wall of the chamber by a hook and loop fastening material. After use, the bag is tied for disposal.

The document of patent U.S. Pat. No. 6,146,875 discloses a method for producing a liquid culture medium, such as, for example, culture broth or a diluent solution, for incubating or culturing a sample. The invention incorporates the use of a bag having a reinforced base, which allows the bag to stand alone without any external support. In the preferred embodiment, the reinforced bag is comprised of a pair of thin film plastic sheets that lay one on top of the other when the bag is not filled with a culture medium. The lower portion or region of the sheets are connected together and closed to form the reinforced base. The side edges of the sheets are bonded directly to each other without the use of a reinforcement. The upper part of the sheets may be spread apart in order to fill the bag. In accordance with one embodiment of the invention, the liquid culture medium is sterilized before it is placed in a presterilized bag. Presterilization of the liquid culture medium is done by placing the medium in an autoclave and subjecting it to sterilization temperatures and pressures. In accordance with a preferred embodiment, the bag is presterilized using a separate procedure of irradiation to kill any microorganisms. The presterilized bag is then filled with the presterilized culture medium in a clean room. Since the bag is reinforced and self-supporting, it stands up by itself when filled and can be moved from place to place like a rigid container. The prefilled and presterilized bag is then sent to a lab to incubate the sample. The culture sample is added to the culture medium by opening the bag and subsequently sealing it. Preferably, the bag is designed to have a re-sealable opening in the form of a “zip-lock” closure, although other ways of sealing the bag may work just as well. After sealing, the sample is incubated in accordance with known incubation procedures, according to sample type. An alternative method of resealing the bag is to use a closure wire instead of a “zip lock” closure. The closure wire is connected to the upper part of one of the sheets, and has a length that exceeds the width of the bag. After the culture medium is added to the bag, the upper portions of the pair of sheets, above the wire closure, are bonded together to form an airtight seal. When the bag arrives at the laboratory, the upper part of the bag is opened, breaking the airtight seal, allowing a culture sample to be inserted into the bag. Afterwards, to reseal the bag, the upper part of the bag is folded or rolled about the closure wire, and then the ends of the closure wire are wrapped around the rolled upper portion, to prevent it from unrolling. In an alternative embodiment, the bag and medium are sterilized in the same step by radiation. In this embodiment, a non-sterile culture broth is added to a non-sterile bag and then subjected to radiation by gamma rays or electrons to render the product sterile. A second advantage of the invention is that the use of a clear, reinforced bag allows visual inspection of the culture medium and sample at any time during the culturing or test process. For example, a user can easily determine whether the contents of a prefilled and sterilized bag have been contaminated at any point in time during transport or storage. Normally, a prefilled and presterilized bag will be quarantined for a period of time as a manufacturer's test for sterility prior to supplying it to a laboratory. Quarantine will reveal contamination by visual inspection. If the bag passes quarantine, then it is deliverable to the laboratory as a “ready-to-use” unit for incubation purposes. Similarly, after the sample is placed in the bag and incubated, the user can visually inspect it for microorganism growth. A third and most important advantage of the invention is that it significantly reduces laboratory costs. There is now a significant market relating to supplying labs with prefilled and presterilized culture media in ready-to-use, rigid bags. However, there is no mechanism for easy recycling of the containers back to the supplier for reuse. Consequently, the bags tend to be used once and then disposed of by the laboratory. This creates unnecessary work and the incremental cost of the bag is significant relative to what is supplied to the laboratory.

Replacing rigid bags with reinforced bags retains all the advantages of rigid bags and provides additional advantages relating to significantly lower costs per bag, with a reduction in total mass and volume of waste material.

It can be observed that the container bags for urine and other body liquids, used in hospitals, which are already known in the current state of the art, do not envisage a means to allow the gel, contained inside the bag, to assume a regular cylindrical-stem form, in order that, with the assistance of a scale located on the outside of the bag, it is possible to confidently check the loss of liquid of the patient, allowing the undertaking of the appropriate body fluid balance of the patient, precisely controlling the volume of liquid to be administered, according to the ascertained loss.

Therefore, the proposition becomes desirable of a disposable bag to collect urine and other body liquids, with a simple construction and low cost, which allows comfortable and secure use, ensuring, after use, the appropriate hermetic sealing, allowing the gelled content inside the said bag to assume a regular cylindrical-stem form, facilitating, through the metric scale located outside the bag, the accurate establishment of its correspondence to the volume of liquid lost by the patient, for it to be possible to undertake the appropriate body fluid balance of the patient.

### SUMMARY OF THE INVENTION

With the intention of overcoming the inconveniences of the current techniques, a disposable bag is proposed to collect urine and other body liquids, designed preferentially, although not exclusively, for hospital use. Its body is formed from front and rear plates made from flexible plastic, with a malleability appropriate for this purpose, in a preferably rectangular format. Its side edges are bonded together, by means of any appropriate adhesion process, forming side flaps that extend along the entire longitudinal extension of the body of the bag. It also has a lower closure formed by a flexible plastic plate, compatible with the material used in the manufacture of the front and rear plates of the bag, in a preferentially circular format, establishing the peripheral edge, which is bonded by means of any appropriate adhesion process, to the lower edges of the front and rear plates of the bag, forming reinforced lower flaps that extend along the entire peripheral extension of the lower edge of the bag, providing the appropriate structure of the bag, allowing it to remain stable in the vertical position, also establishing, after the absorption of the liquid, a cylindrical-stem format of the gelled content inside the bag, in order that, with the assistance of the scale located vertically on the outside of the bag, it is possible to establish the correspondence of the height of the cylindrical-stem body formed by the gelled content inside the bag and the volume of liquid lost by the patient, allowing the accurate measurement of the body fluid balance of the patient.

The lower closure plate of the bag can be folded transversally, allowing, by the overlapping of the front and rear plates, the flattening of the bag, when not in use, for its individual packaging in a wrapper to guarantee its asepsis.

The lower closure plate can assume any form that provides the obtainment of a regular format to the gelled content of the bag, which allows the correlation between its height to the volume of liquid absorbed by the patient.

The disposable bag to collect urine and other proposed body liquids can be used by men and women, independent of the age range. However, in order to provide an anatomical design that is more favorable to women, its flexible mouth, made from Ethylene-Vinyl Acetate (EVA) or any other material appropriate for this purpose, as well as being anatomical, has one extremity longer than the other, and can also be made available in colored and/or scented versions.

The disposable bag to collect urine and other proposed body liquids also provides a closure of its upper opening by means of a seal, of the Ziploc® type, which extends along the entire transversal extension of the bag. It can be opened and closed as many times as necessary, impeding the infiltration of humidity or access to the crystals, before or after its use, as well as the emptying of its content.

The disposable bag to collect urine and other proposed body liquids can also be made in a material that allows recycling, composting, degradation and other forms that do not harm the environment.

The disposable bag to collect urine and other proposed body liquids can be made available in different sizes and capacities.

The disposable bag to collect urine and other proposed body liquids can also be used as a personal convenience item, overcoming the lack of availability of bathrooms.

### BRIEF DESCRIPTION OF THE DRAWINGS

For a better understanding of the disposable bag to collect urine and other proposed body liquids reference is made to the attached figures, in such a way that they can be entirely reproduced by the appropriate technique, allowing the full characterization of its functionality. However, these drawings are merely illustrative and can vary, provided they do not avoid the proposed functional principle, and where:

FIG. 1 illustrates an upper perspective view of the packaging that contains one unit of the proposed disposable bag;

FIG. 2 illustrates a perspective view of the disposable bag, extracted from the individual packaging, in the folded position;

FIG. 3 illustrates a perspective view of the disposable bag being unfolded for use;

FIG. 4 illustrates a perspective front view of the disposable bag in use;

FIG. 5 illustrates a perspective rear view of the disposable bag in use;

FIG. 6 illustrates a perspective front view of the disposable bag after absorption of the liquid; and

FIG. 7 illustrates a perspective rear view of the disposable bag, after absorption of the liquid, stabilized in the vertical position.

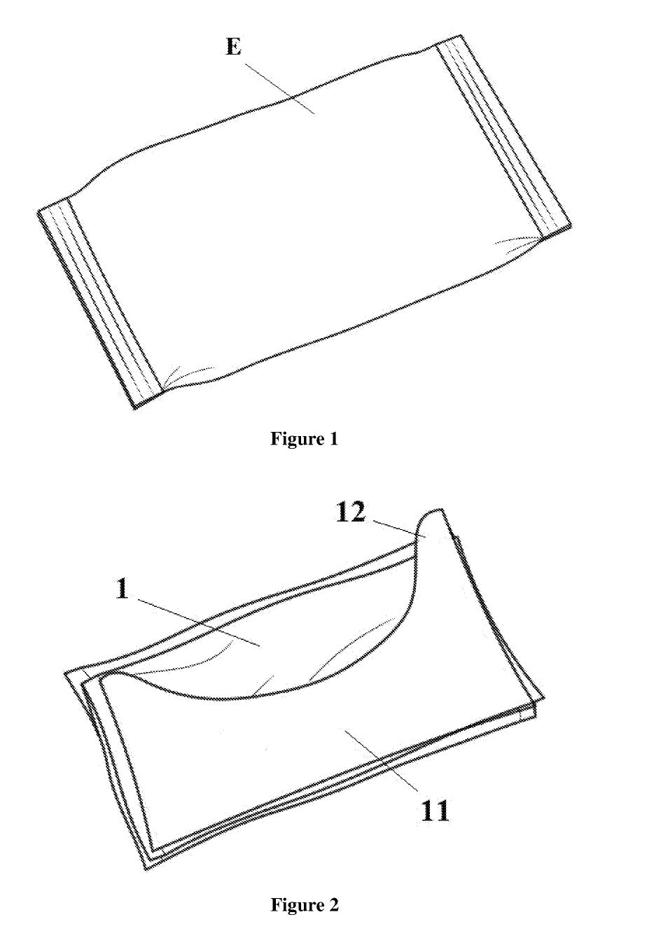

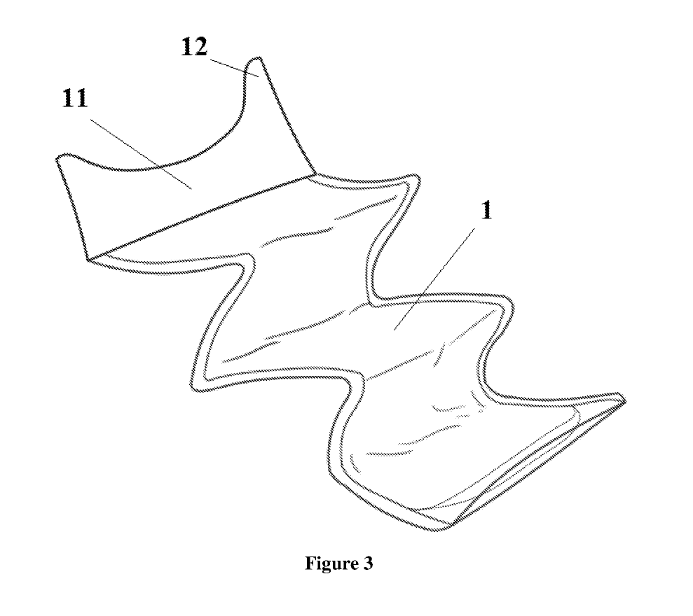

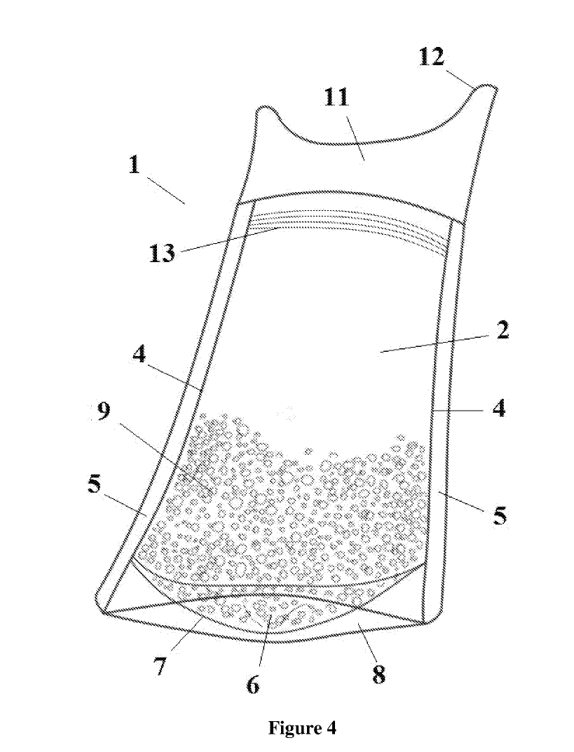

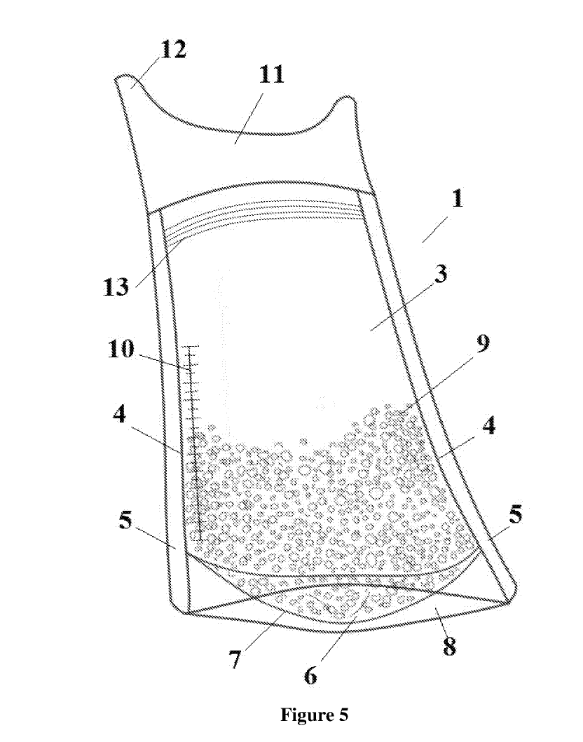

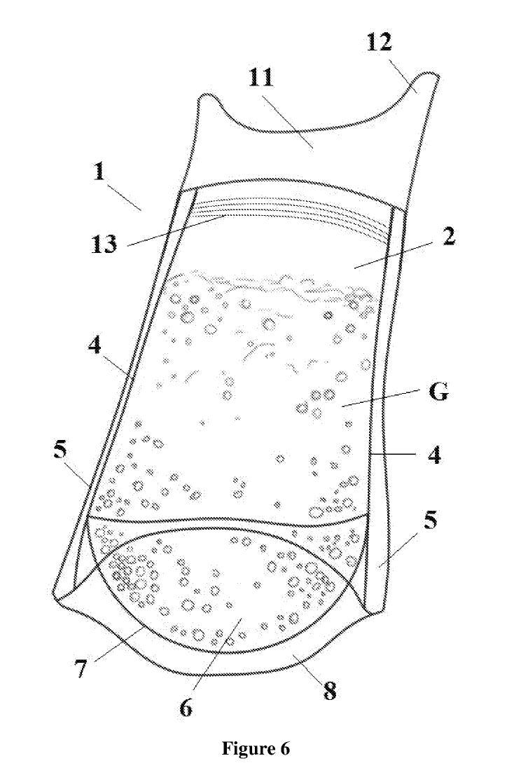

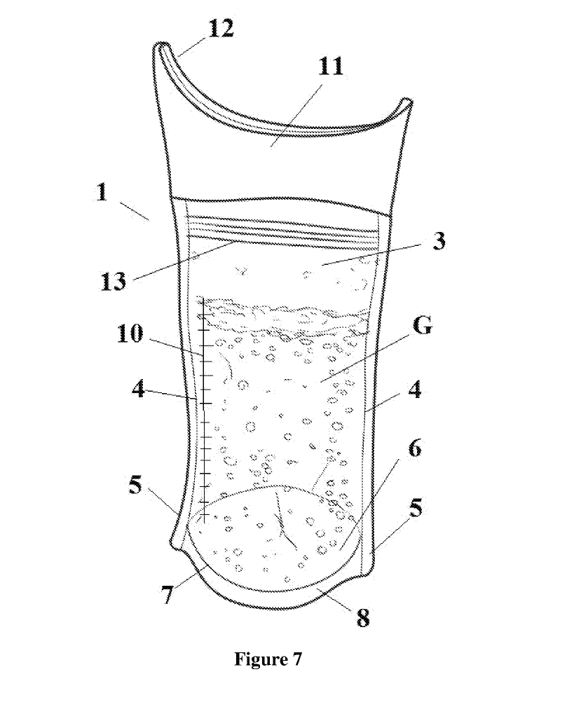

### DETAILED DESCRIPTION OF THE INVENTION

In accordance with that illustrated in the attached figures, the disposable bag to collect urine and other proposed body liquids is supplied in an individual packaging (E), the body (1) of the bag formed by front (2) and rear (3) plates, made from flexible plastic, with a malleability appropriate for this purpose, in a preferably rectangular format. Its side edges (4) are bonded together, by means of any appropriate adhesion process, forming side flaps (5) that extend along the entire longitudinal extension of the body of the bag. It also has a lower closure formed by a flexible plastic plate (6), compatible with the material used in the manufacture of the front and rear plates of the bag, in a preferentially circular format, establishing the peripheral edge (7), which is bonded by means of any appropriate adhesion process, to the lower edges of the front and rear plates of the bag, forming reinforced lower flaps (8) that extend along the entire peripheral extension of the lower edge of the bag, providing the appropriate structure of the bag, allowing it to remain stable in the vertical position. It has gel crystals (9) inside, which, after absorption of the liquid, establish a cylindrical-stem format of the gelled content (G) inside the bag, in order that, with the assistance of the scale (10), located vertically on the outside of the bag, it is possible to establish the correspondence of the height of the cylindrical-stem body formed by the gelled content inside the bag and the volume of liquid lost by the patient, allowing the accurate measurement of the body fluid balance of the patient.

The lower closure plate of the bag has a transversal folding line, allowing, by the overlapping of the front and rear plates, the flattening of the bag, when not in use, for its individual packaging in a wrapper to guarantee its asepsis.

The disposable bag to collect urine and other proposed body liquids can have a scale located on its front and/or rear side.

The disposable bag to collect urine and other proposed body liquids can be used by men and women. In order to provide an anatomical design that is more favorable to women, its flexible mouth (11), made from a rubberized material, of the EVA type or any other material appropriate for this purpose, has one extremity (12) longer than the other, and can also be colored and/or scented.

The disposable bag to collect urine and other proposed body liquids provides an upper closure by means of a seal (13), of the Ziploc® type, which extends along the entire transversal extension of the bag, impeding the infiltration of humidity or loss of gel crystals and the emptying of its content.

The disposable bag to collect urine and other proposed body liquids can be made in a material that facilitates its recycling, composting and degradation, without harming the environment.

The disposable bag to collect urine and other proposed body liquids can also be used when in movement, such as, for example, inside ambulances.

The disposable bag to collect urine and other proposed body liquids minimizes the propagation of infectious and contagious diseases and odors.

The disposable bag to collect urine and other proposed body liquids can be made available in different sizes and capacities.

The disposable bag to collect urine and other proposed body liquids can also be used as a personal convenience item, overcoming the lack of availability of bathrooms.

1. A disposable bag to collect urine and other body liquids comprising:
a body (1) formed by a front plate (2) and a rear plate (3), with side edges (4) bonded together, forming side flaps (5), with a lower closure formed by a plate (6), establishing a peripheral edge (7), forming lower reinforced flaps (8), also having gel crystals (9) inside the body (1),
wherein after the absorption of the liquid, the gelled content (G) inside the body (1) establishing a regular cylindrical-stem format.

2. The disposable bag in accordance with claim 1, wherein the bag being supplied in an individual packaging (E).

3. The disposable bag in accordance with claim 1, wherein the front (2) and rear (3) plates are being made from flexible plastic with a malleability appropriate for this purpose.

4. The disposable bag in accordance with claim 1, wherein the front (2) and rear (3) plates have a rectangular format.

5. The disposable bag in accordance with claim 1, wherein the side edges (4) of the front (2) and rear (3) plates are bonded together by an adhesion process.

6. The disposable bag in accordance with claim 1, wherein the side flaps (5) extend along the entire longitudinal extension of the body of the bag.

7. The disposable bag in accordance with claim 1, wherein the lower closure plate (6) is made from flexible plastic with a malleability appropriate for this purpose.

8. The disposable bag in accordance with claim 1, wherein the lower closure plate (6) have a circular format.

9. The disposable bag in accordance with claim 1, wherein the lower closure plate (6) provides a regular format to the gelled content after the absorption of the liquid.

10. The disposable bag in accordance with claim 1, wherein the lower closure plate (6) have a transversal folding line.

11. The disposable bag in accordance with claim 1, wherein the lower closure plate (6) establishes a peripheral edge (7) and wherein the peripheral edge (7) of the lower closure plate (6) is being bonded by means of an appropriate adhesion process to the lower edges of the front and rear plates of the bag.

12. The disposable bag in accordance with claim 1, wherein the lower reinforced flaps (8) extend along the entire peripheral extension of the lower edge of the bag.

13. The disposable bag in accordance with claim 1, wherein the side flaps (5) and the lower reinforced flaps (8) provide an appropriate structure of the bag to allow the disposable bag to remain stable in the vertical position.

14. The disposable bag in accordance with claim 1, further including a scale (10) located vertically on the outside of the bag.

15. The disposable bag in accordance with claim 1, wherein the height of the gelled content (G) inside the bag allows, with the assistance of the scale (10), the establishment of correspondence with the volume of liquid lost by a patient.

16. The disposable bag in accordance with claim 1, wherein the body of the disposable bag is capable of being flattened, by the overlapping of the front and rear plates, when not in use.

17. The disposable bag in accordance with claim 1, wherein the disposable bag is capable of being used by men and women.

18. The disposable bag in accordance with claim 1, further including a flexible mouth (11), when used by women is made from a rubberized material of the EVA type.

19. The disposable bag in accordance with claim 18, wherein the flexible mouth (11) having one extremity (12) longer than the other.

20. The disposable bag in accordance with claim 18, wherein by the flexible mouth (11) capable of being colored and/or scented.

21. The disposable bag in accordance with claim 1, further including an upper closure by a seal (13), of the Ziploc® type, which extends along the entire transversal extension of the bag.
